(19) United States
(12) Patent Application Publication
(10) Pub. No.: US 2003/0130741 A1
(43) Pub. Date: Jul. 10, 2003

(54) Hip prosthesis

(76) Inventor: Derek James Wallace McMinn, Stourbridge (GB)

Correspondence Address: CANTOR COLBURN, LLP, 55 GRIFFIN ROAD SOUTH, BLOOMFIELD, CT, 06002

(21) Appl. No.: 10/041,799

(22) Filed: Jan. 7, 2002

(51) Int. Cl. A61F 2/36; A61B 17/16; A61B 17/56; A61B 17/92

(52) U.S. Cl. 623/23.14; 606/80; 606/81; 606/89; 606/99

(57) ABSTRACT

A method of hip resurfacing in which a first incision is made at a patient's hip joint, and a second incision is made at the other side of the patient's thigh, so that a stem part of an instrument or of an implant component applicator can be inserted through said second incision and along the inside of the femoral neck, and an operative part of the instrument, and/or an implant component, can be inserted through the first incision. The operative part and the stem part of the instrument can be releasably connected, as can the stem part of the implant component and the implant component, either directly or indirectly. The instruments can be an acetabular reamer, a sleeve cutter, a chamfer cutter or an impactor cap. The implant component is a femoral component.

This invention relates to artificial hip joints, particularly to a method of resurfacing the femoral head and the acetabulum to provide such an artificial joint, and also to components and instruments for use in the method.

Resurfacing of arthritic hips, commonly carried out on younger patients, is presently performed as an open procedure, using an implant comprising an acetabular component and a mating femoral component. Although the implant is normally small, the surgical approach, of necessity, has to be large in order to gain access to the femoral head and neck, to allow jigging systems to be applied to the femur for correct femoral alignment, and to allow displacement of the femoral head to gain access to the acetabulum for acetabular preparation and component insertion. The implant is inserted through a large surgical incision at one side of the hip, for example by initially making a straight incision in the skin on the posterior edge of the greater trochanter. Disadvantages of such a large incision are the amount of trauma suffered by the patient and the consequent relatively long rehabilitation period and post-operative hospital stay.

An object of the invention is to overcome this disadvantage.

According to a first aspect of the invention there is provided a method of hip resurfacing comprising the steps of making a first incision at the hip joint, and making a second incision at an outer side of the thigh, inserting a stem part of an instrument, or an implant component applicator stem, through said second incision and along the inside of the femoral neck, and inserting an operative part of the instrument, and/or an implant component, through said first incision, the operative part being releasably connected to said stem part, and the implant component being releasably connected directly or indirectly to said stem, or acted upon by the or an instrument.

Preferably the releasable connection of the operative part of the instrument or the implant component with the stem part or stem respectively is a screw-thread. Accordingly, for example, after the use of one instrument has been completed, its operative part is unscrewed from the stem part and a different operative part is screwed onto the stem part. The stem part may be power driven and the stem may be formed with a slaphammer. The stem may be arranged to be releasably connected to a further component of the implant, such as a rod-like stem, which itself is screw-threadedly connected to the main implant component, so that the main implant component is indirectly connected to the applicator stem. Alternatively the main femoral implant component can have said rod-like stem integral therewith, with the free end of the rod-like stem having means for releasably connecting it to said stem. The connections can be other than screw threads. In particular although the rod-like stem would probably be releasably connected to the main cup-shaped femoral implant component part by screw thread means, the implant component applicator stem would probably be releasably connected to said rod-like stem by other means, for example an L-shaped slot engaging a reciprocal protuberance. In one embodiment the implant component is an acetabular cup which is acted on by an instrument in the form of an impactor cap to fix the acetabular cup in position.

According to a second aspect of the invention there is provided an instrument for use in hip resurfacing comprising a stem part and a releasably connectable operative head part, portions of the releasable connection being provided at or adjacent an end of the stem part and at said head part respectively.

Preferably said operative part is an acetabular reamer, a sleeve cutter, a chamfer cutter or an impactor cap. Particularly with an acetabular reamer, the shaft could be releasably connected to a member which itself is connected to/within the operative head part of the reamer. The member is, for example, a spider-like connector.

According to a third aspect of the invention there is provided a femoral implant component for use in hip resurfacing, the component comprising a generally cup-shaped part having a part-spherical outer surface and connection means for directly or indirectly connecting the component, in use, with a rod/stem of an implant component applicator.

Preferably in both the second and third aspects, the direct releasable connection of the rod/stem part to the head part or of the rod/stem to said cup-shaped part is a screw-threaded connection. In this latter case, the screw thread of the cup-shaped part is at or adjacent an inner surface thereof.

The invention will now be described, by way of example, in which:

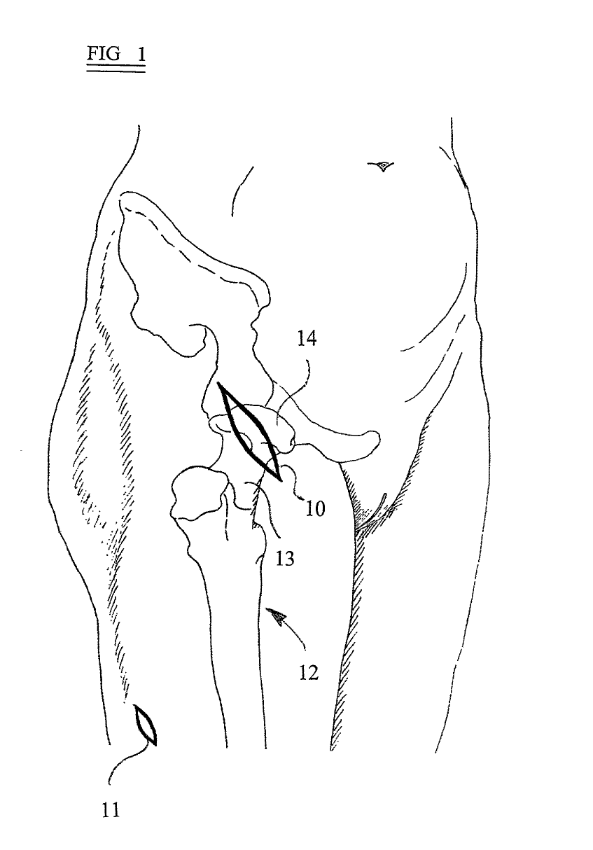

FIG. 1 is a schematic view showing a first stage of a method of hip resurfacing of the invention, with incisions being made at the front of the hip joint and at an outer side of a patient's thigh respectively,

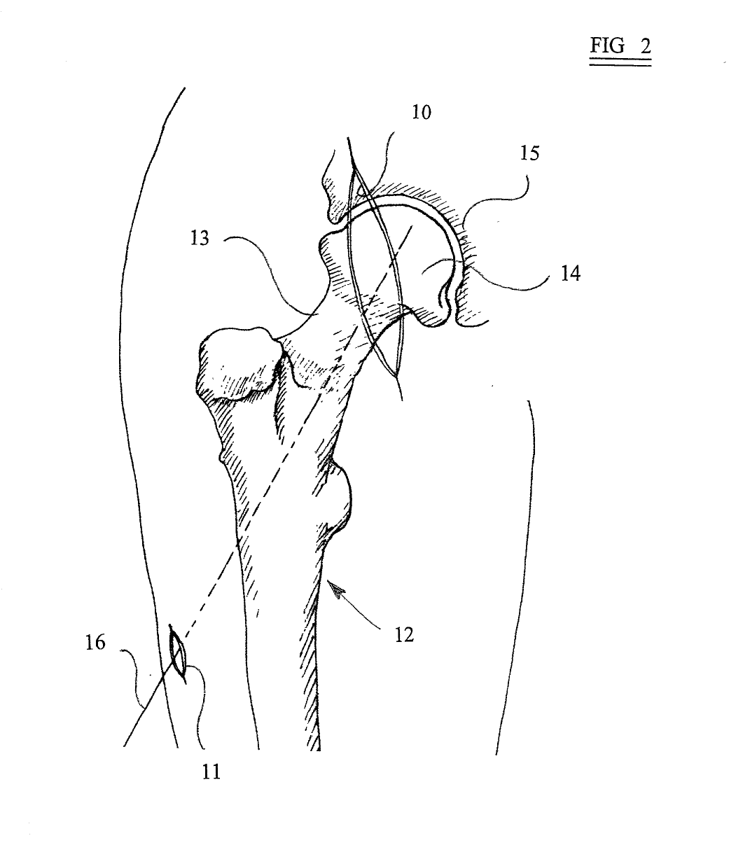

FIG. 2 is an enlarged view of part of FIG. 1, showing insertion of a guide wire through the incision made at the outer side of the thigh,

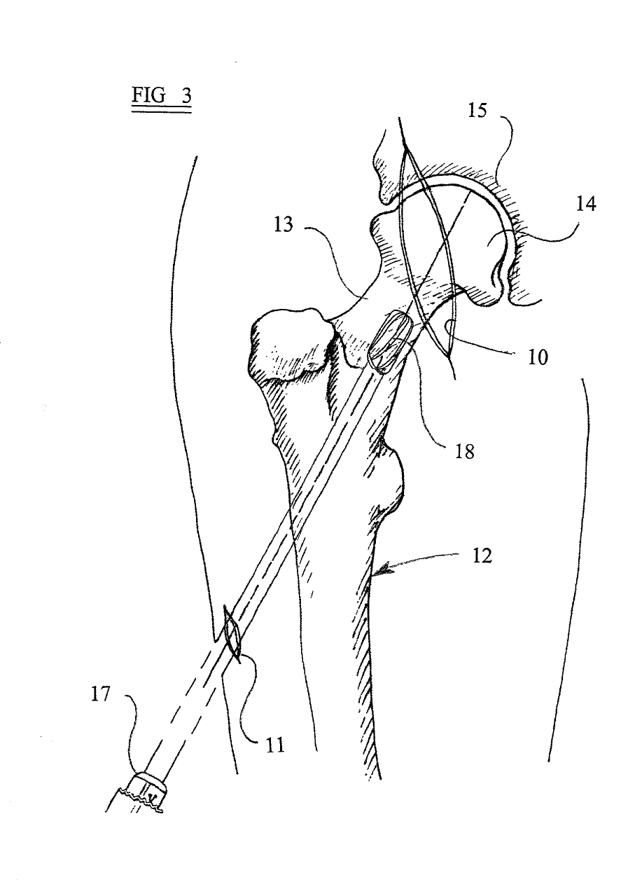

FIG. 3 is a view like FIG. 2, showing the insertion of a stem part of an instrument through said incision at the outer side of the thigh,

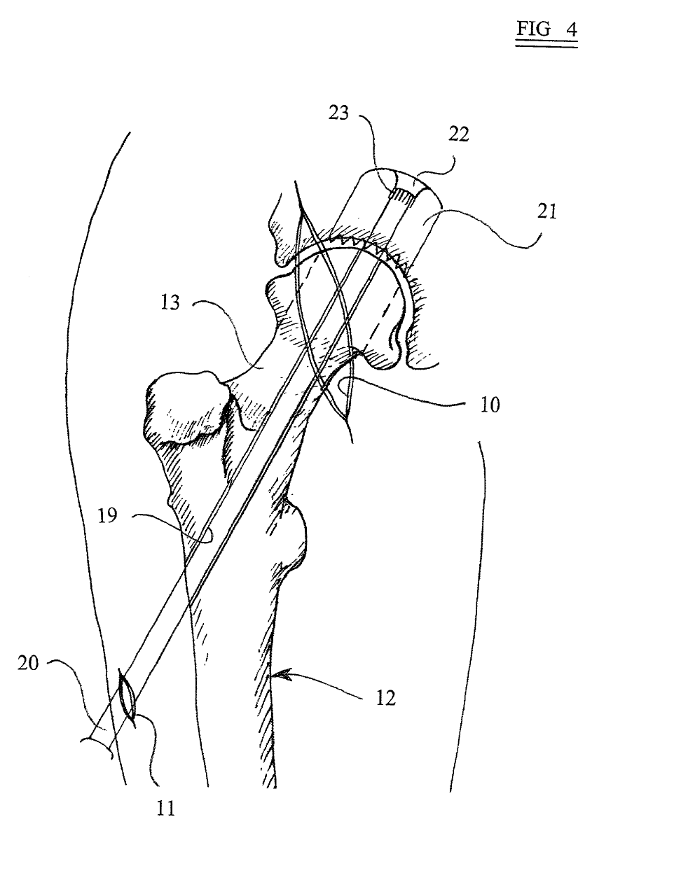

FIG. 4 is a view like FIG. 3, showing the use at the femoral head of a sleeve cutter inserted through the incision at the front of the hip joint,

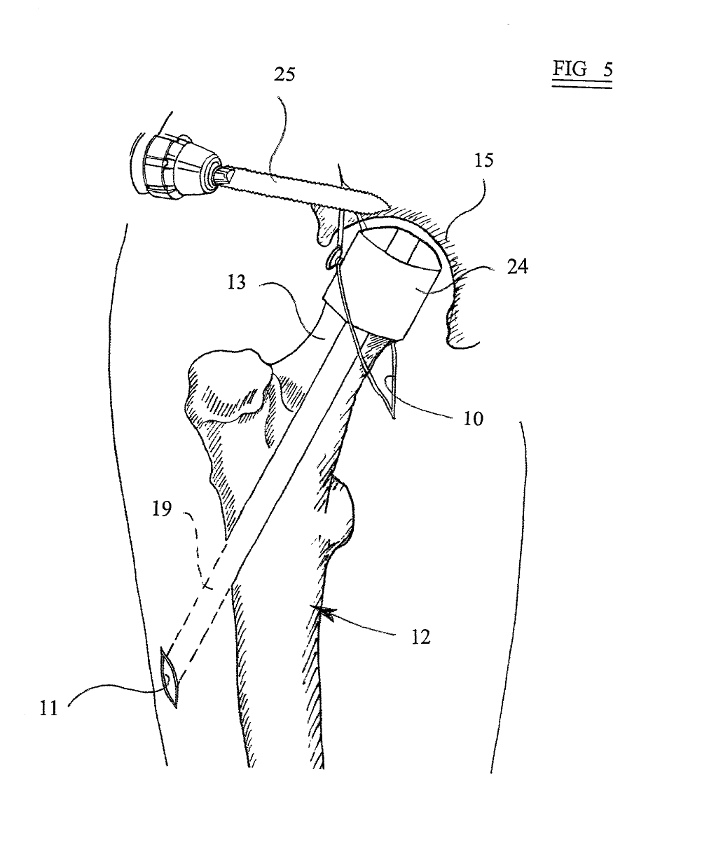

FIG. 5 shows the use of a sleeve resection guide at the femoral head following the use of the sleeve cutter,

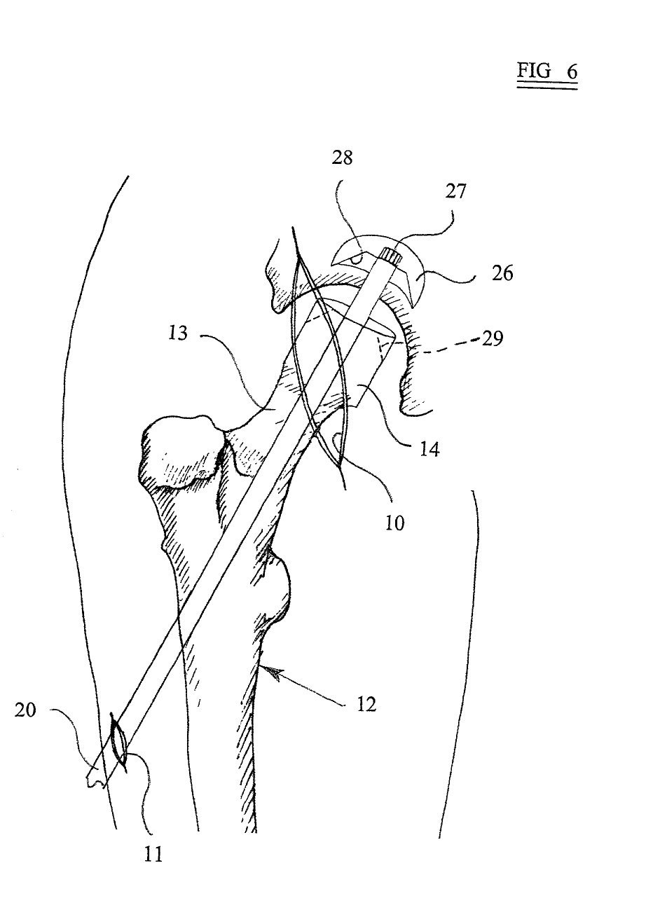

FIG. 6 shows the use of a chamfer cutter at the femoral head following the use of the sleeve resection guide,

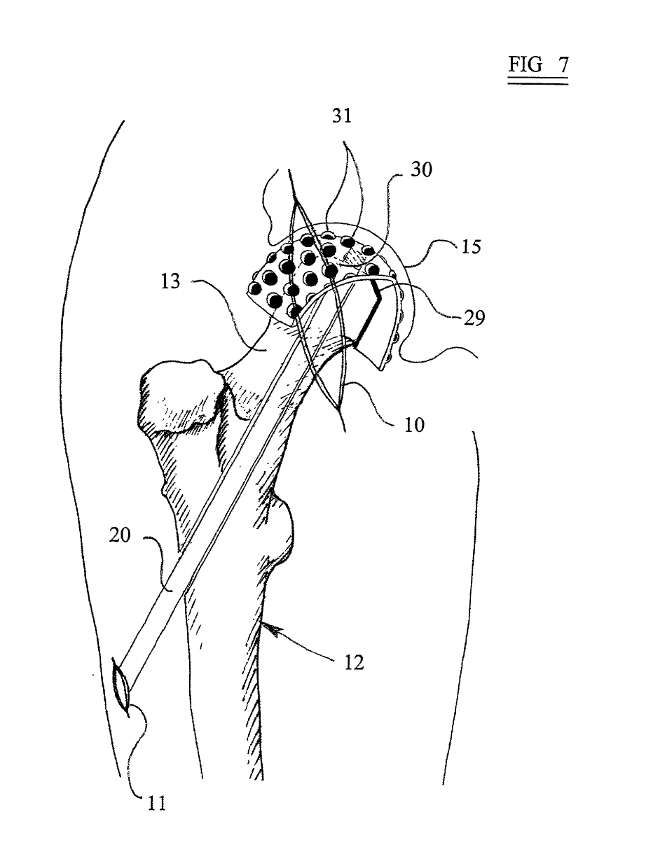

FIG. 7 shows the use of an acetabular reamer, following completion of femoral head preparation,

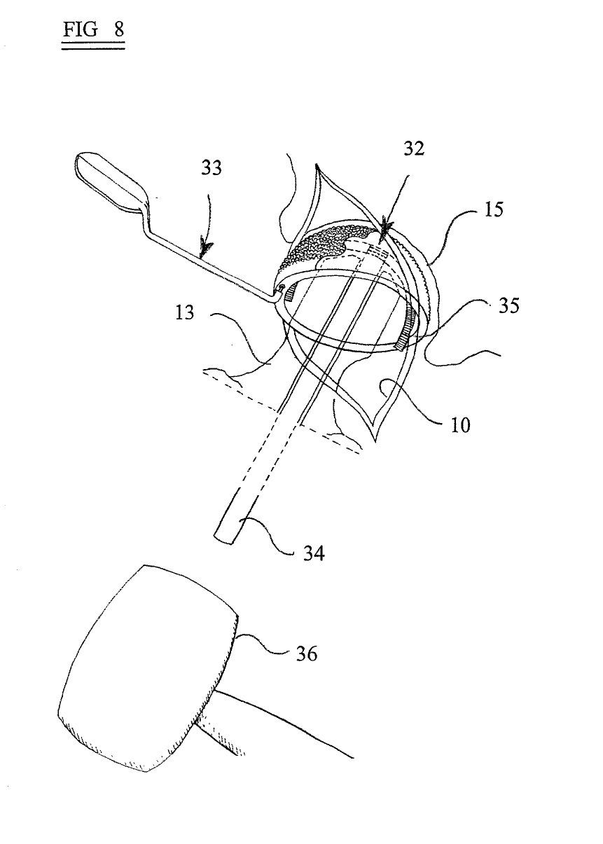

FIG. 8 shows schematically the fitting of an acetabular implant component,

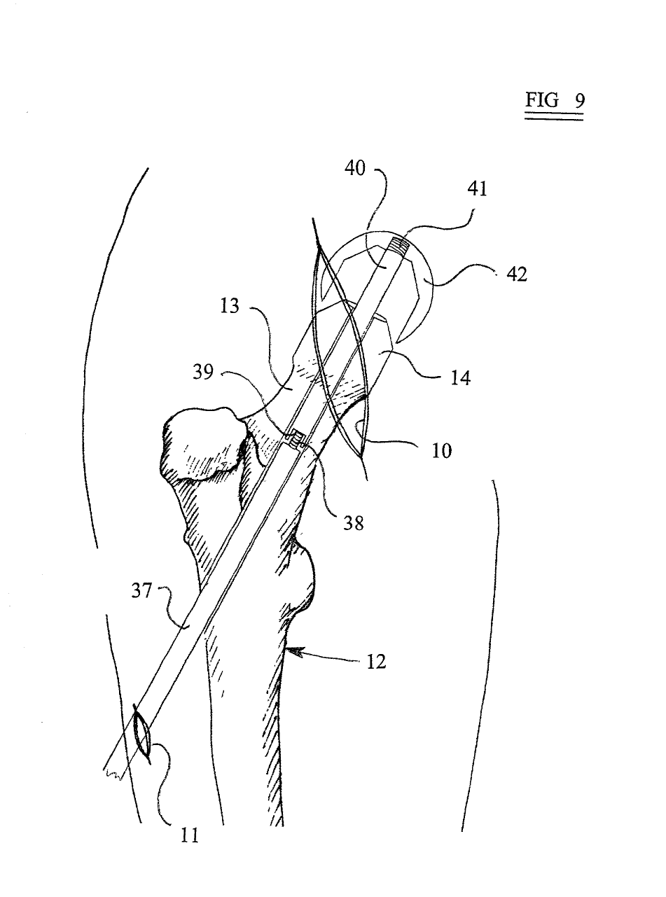

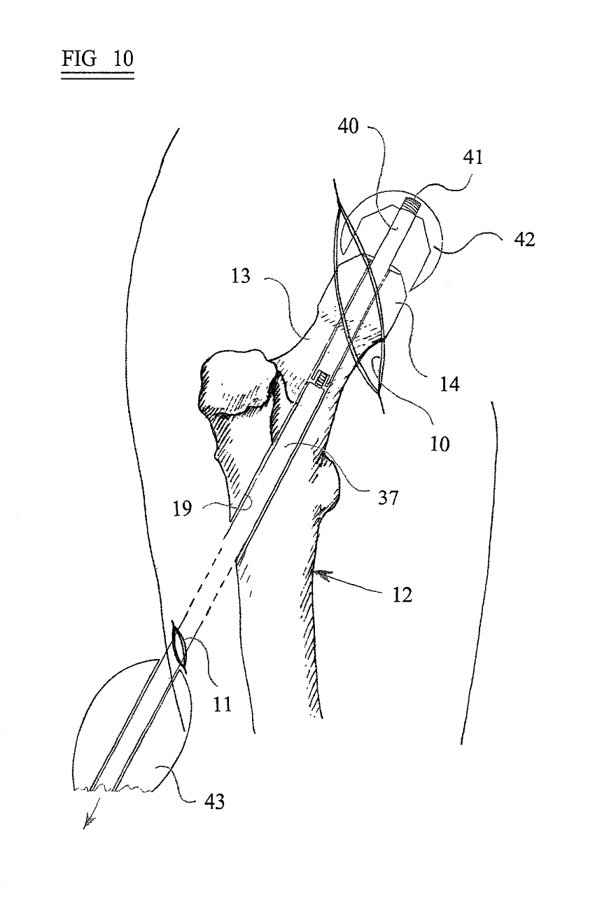

FIGS. 9 and 10 schematically show successive stages in the fitting of a stemmed femoral implant,

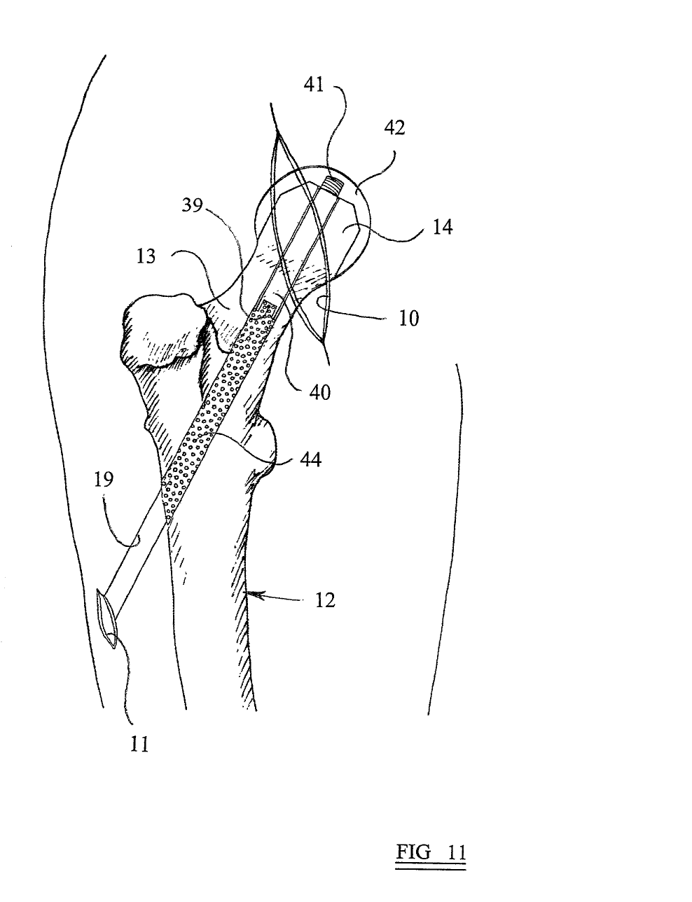

FIG. 11 schematically shows the femoral implant component in a resurfaced hip joint prior to closure of the surgical incisions, and

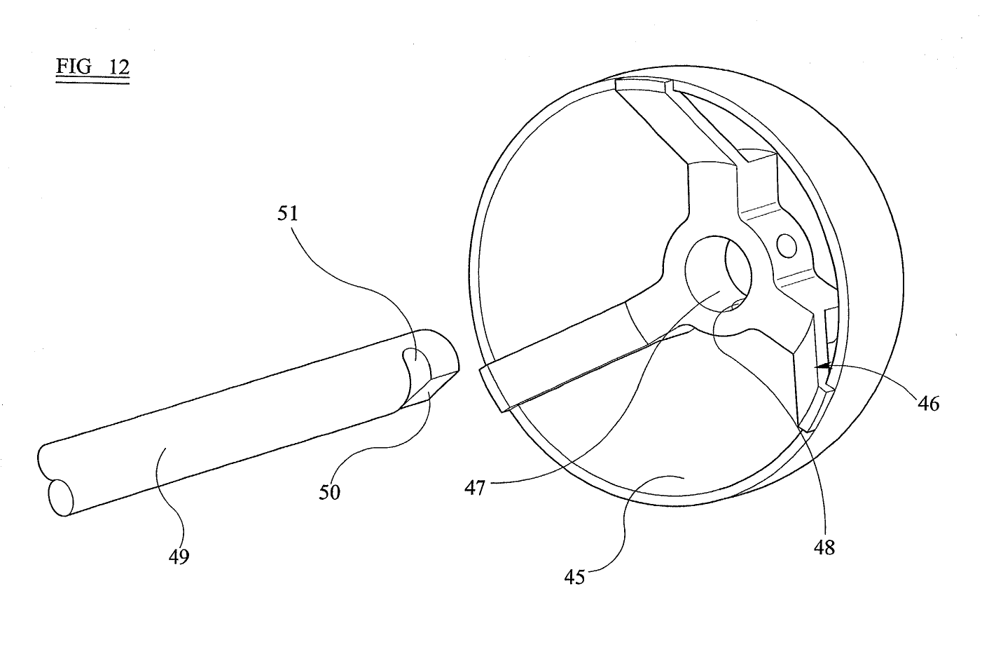

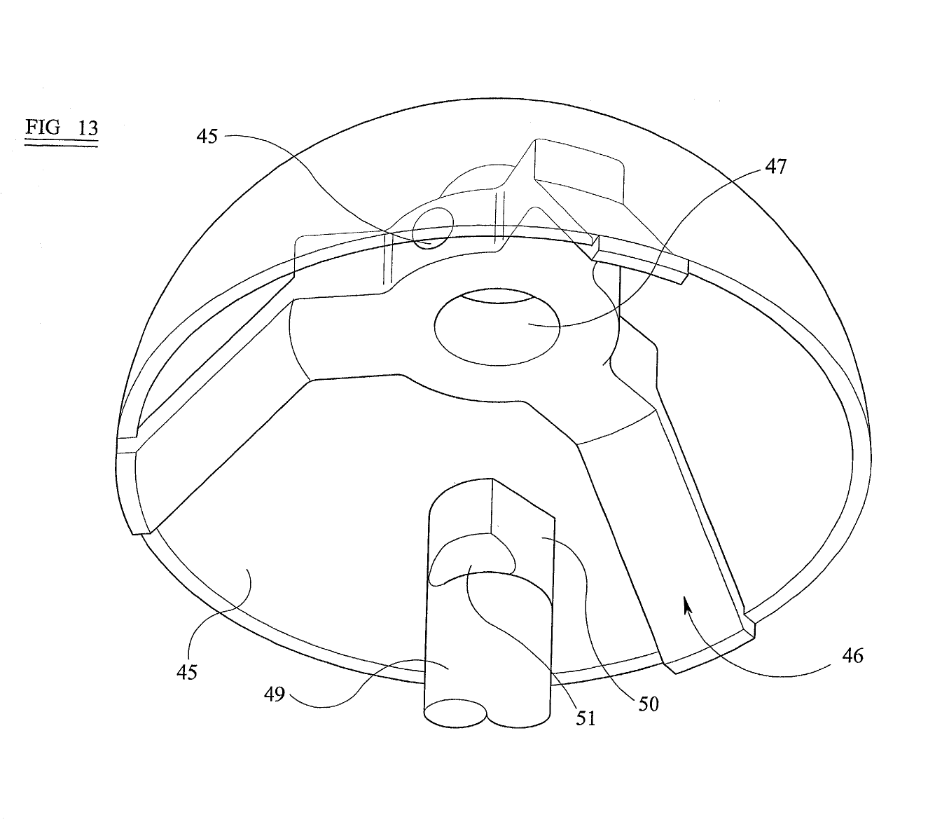

FIGS. 12 and 13 are respectively schematically a perspective view and an enlarged perspective view of an alternative releasable connection between a rod part and an acetabular reamer.

The first stage in the surgery for carrying out hip resurfacing according to the method of one aspect of the invention is as illustrated in FIG. 1. With present hip resurfacing techniques, a rather large surgical incision is made at one side of the hip, as previously mentioned above. In contrast, the first stage of the method of the invention is to carry out the whole operation through a much smaller incision 10, for example a 6 cm incision, at the ‘front’ of the hip joint, with a further small incision 11, for example of 2 cm, at the outer side of the thigh. The incision 10 can be disposed anteriorly (as illustrated), posteriorly or centrally of the hip joint. Thus instead of forming in the hip a large opening where all the surgical instruments and implant components are inserted directly into or onto the bone, the provision of the two smaller incisions 10 and 11, as will be described, enables a stem or rod, shown in FIG. 3, to be inserted through the incision 11 and up the inside of the femoral neck, and cutter or reamer parts of the surgical instruments and the implant components of the resectioning to be inserted via the incision 10. As will be described, the stem or equivalent is adapted to be engaged with operative parts of the instruments or with the femoral implant component so as to allow the various surgical steps of the hip resurfacing to take place.

FIGS. 1 and 2 both show a conventional femur 12 having a neck 13 and head 14. FIG. 2 shows in greater detail the acetabulum 15 with which the femoral head 14 mates to form the hip joint.

Once the incisions 10 and 11 have been made as shown in FIG. 1, a guide-wire 16, shown in FIG. 2, is inserted through the incision 11 into the lateral aspect of the femur and up into the femoral head and neck, with the desired position obtained using either an external alignment jig, a navigation system, or x-ray control. Following insertion of the guide-wire, the femoral head 14 is dislocated from the acetabulum in dependence on which approach to the hip has been made. Since in the illustrated embodiment the approach is anterior, the femoral head would be dislocated anteriorly.

The guide-wire 16 is then over-drilled, producing a canal, typically of 8.5 mm in diameter, up the femur 12, femoral neck 13, and exiting through the zenith of the femoral head 14. FIG. 3 shows a typical drill 17 with cutting bit 18. Once the canal 19 has been completed, the drill 17 is removed and preparation of the femoral head is then commenced.

Whilst the surgical method may involve the use of some instruments and implant components which are of a size and shape which enable them to be inserted through the incision 10, some cannot be inserted in complete form through said incision, and a feature of another aspect of the invention is that such instruments/implant components are formed as an operating or main part, which is inserted through the incision 10, and releasably connectable to at least one rod or stem which is inserted through the incision 11 and along the canal 19.

Accordingly the first step in femoral head preparation is the insertion of a drive rod 20 up the canal 19 in the femur. An appropriately sized sleeve cutter 21 of generally hollow cup shape is inserted through the incision 10 and releasably secured to the end of the drive rod 20, which extends out of the top of the femoral head 14. This releasable connection can be of any suitable form, but, in the embodiment illustrated, is typically a screw-threaded connection, with a threaded end of the drive rod 20 being engaged with an internal thread of a socket or boss 22 formed at the centre of the inside of the cup-like cutter 21, as shown in FIG. 4. Once the inserted cutter has been threadedly engaged onto the end of the drive rod 20, this rod is driven by any suitable power means and the cutter advanced down onto the femoral head 14 so that the periphery thereof is thus resected. The cutter is then unscrewed and removed via the incision 10, with the drive rod 20 being removed via the incision 11. The threaded end of the rod 20 is schematically indicated at 23 in FIG. 4.

The next method step, shown in FIG. 5, involves the application of a sleeve resection guide 24 to the periphery of the femoral head, the guide 24 being inserted through the incision 10. An appropriate amount of zenith of the femoral head 14, as determined by the positioning of the guide 24, is then resected using a powered cutting blade 25, thereby maintaining the patient's correct leg length. After completion, the guide 24 is removed through the incision 10.

The drive rod 20 is then again inserted through the incision 11 and up along the canal 19 so that its threaded end 23 extends from the top of the resected femoral head. An appropriately sized chamfer cutter 26 is then inserted through the incision 10 and attached to the end of the drive rod. As with the cutter 21, the releasable connection can be of any suitable form, but preferably, with the threaded end 23 described, the chamfer cutter has at the centre of its interior a threaded socket 27, schematically shown in FIG. 6. As can be seen from this Figure, the cutter 26 has a continuous chamfered cutting surface 28 extending inwards from its outer periphery. Accordingly, in use, the drive rod 20 is moved down the canal 19 so that the cutter 26 is brought down onto the femoral head so that when the drive rod 20 is powered to rotate, the cutter 26 similarly rotates and cuts the femoral head to provide a chamfer thereon, indicated schematically at 29. Once the uppermost part of the femoral head is correctly chamfered as at 29, the cutting is stopped and the drive rod 20 and cutter 26 are unscrewed to release the connection between them, the cutter 26 then being removed through the incision 10, and the rod 20 removed, if necessary, through the incision 11. It will be appreciated that in some instances the drive rod 20 may be suitable for connection to various instrument parts, whilst in other embodiments differently sized drive rods may be required, or ones having different end connections to the operative or main parts of the instruments respectively.

Once the chamfering of the femoral head shown in FIG. 6 has been carried out, the femoral head preparation is completed.

Attention is now turned to acetabular preparation. As shown in FIG. 7, an acetabular reamer 30 is inserted through the incision 10. Such a reamer is typically of hollow cup-like form having a generally hemispherical outer surface, with its hollow interior having a central internally threaded socket for engagement with the complementary threaded end 23 of the drive rod 20. Thus after insertion through the incision 10, the reamer 30 and the rod 20 are screwed together. The rod 20 is then powered to rotate, and the reamer is advanced upwardly to the acetabulum so that reaming can take place. This process is carried out firstly with an appropriately sized acetabular reamer 30 as shown in FIG. 7, and then with increasing sizes of such acetabular reamers, all of which can, for example, have on their respective outer surfaces reaming nodules 31 shown for the reamer 30 in FIG. 7. It will be appreciated that each time a different operative head of the reaming instrument is required, the operative head part is unscrewed from the rod 20, removed through the incision 10, with the next larger required size of operative reaming head being inserted through the incision 10, threadedly engaged with the drive rod end 23 and then rotated and advanced to effect reaming of the acetabulum. Reaming is continued in this way, using successively larger reamers until the acetabulum is reamed to accept the acetabular component of the implant, which is in the form of an acetabular cup 32 shown in FIG. 8.

The internal diameter of the acetabular reamer has to match the prepared shape and size of the femoral head. When the operative part of the reamer is secured to the drive rod over the prepared femoral head, the femoral head and acetabular reamer are reduced into the acetabulum, thereby allowing acetabular reaming. This is quite different from conventional techniques where for hip resurfacing the femoral head is displaced out of the way of the acetabulum thus allowing acetabular reaming, whilst in total hip replacement the femoral head and part of the femoral neck are resected thereby facilitating space for acetabular preparation. By using the described type of acetabular reamer over the prepared femoral head, a minimal surgical exposure to the hip joint can be made and still the acetabulum can be reamed.

FIG. 8 shows how the desired size of acetabular cup 32 is inserted through the incision 10 into the prepared acetabulum, being held by the Surgeon by means of a detachable acetabular cup holder 33. The cup 32 is of generally wholly hemispherical form. Alternative types of cup holder could be used.

An impactor rod 34 is inserted through the incision 11, and up the canal 19 in the femur, femoral neck and femoral head. An impactor cap 35, of generally cup form, is inserted through the incision 10, this cup having a central interior threaded socket part to engage with the complementary external thread on the end of the rod 34, so that the rod and impactor cap can be screwed together. The impactor cap is sized so as to be received in the interior of the acetabular cup 32, so that when the acetabulum is in the correct alignment, the Surgeon can impact the acetabular component, i.e. the cup 32, into the prepared acetabulum to provide a secure initial fixation. Conventionally the impacting can be by way of blows from a mallet 36 onto the end of the impactor rod 34. The acetabular component can be of any suitable form, although it is normally cup-shaped and of a single component. However it could be a composite structure comprising an outer metallic part within which is fitted a plastic insert having a central recess for receiving a part-spherical surface of the femoral implant. Cementing and/or other fixing of the acetabular component can be employed as necessary. Once the acetabular cup 32 is securely fixed in place, the holder 33 is removed and attention is then turned to insertion of the femoral component.

If the impactor cap was removably connected to the acetabular component by a system used currently in known hip resurfacing, namely cables, or other means, then the acetabular component with its fixed impactor cap could be loaded onto the impactor rod in the femoral head and movement of the leg and, therefore, hip could be used to line up the acetabular component in the correct position before impaction. The correct position of the acetabular component is generally held to be 45° to the longitudinal axis of the body when viewed in the frontal plane and 20° anteverted in the coronal plane, i.e. the cup face points 20° forwards.

The femoral component of the implant can be either a cemented or a cementless device. With both the cemented and the cementless variety of femoral component both a stemmed and a non-stemmed version is available. Another aspect of the invention relates to the form of these components which are quite different from the currently available resurfacing femoral components which, with both the stemmed and unstemmed varieties are presently driven onto the prepared femoral head from above in the known resurfacing method described in the introduction, where a single large incision is made. With the present invention, both the stemmed and the unstemmed femoral components are impacted by the use of a detachable femoral prosthesis impactor rod attached either to the stem (in the stemmed variety implant) or the resurfacing cap (in the unstemmed variety of implant). Typically the cemented femoral component cap would be smooth on its inner surface and the uncemented femoral resurfacing cap would have a porous and/or hydroxyapatite coated inner surface for biological fixation.

FIG. 9 shows an uncemented stemmed variety of femoral component being fitted to the prepared femoral head, but, as will be described, the only difference with an unstemmed variety would be the use of a longer impactor rod which screws directly into the femoral component rather than to its stem.

Again due to the small size of the incision 10 it may not be possible to insert and fix conventional femoral implant components in the normal way, i.e. through the incision which provides access to the femoral neck. Accordingly again a releasably connectable rod is inserted through the incision 11 and up the canal 19 so that its threaded end can engage with an internally threaded socket of either the component itself or of a stem component thereof which itself is screw-threadedly engageable with the remainder of the component.

FIG. 9 shows an impactor rod 37 inserted through the insertion 11 and up the canal 19. At its end, which is in the canal 19, this rod is provided with an externally threaded part 38 which is engaged with the complementary internal thread of a socket 39 at one end of a stem 40 which forms part of the femoral implant component. This stem 40 extends from the rod 37 so as to extend out of the canal at the femoral head 14, the stem 40 having its other end externally screw-threaded, as at 41. The main femoral component, in the form of a femoral resurfacing cap 42, is shown in FIG. 9 as being generally of hollow hemispherical form, having a smooth hemispherical outer surface to match the smooth inner hemispherical surface of the acetabular cup 32 to provide the ‘ball and socket’ joint of the resurfaced hip. The interior surface of the cap 42 generally matches the exterior shape of the prepared femoral head 14, i.e. having a straight sided portion leading to a chamfered portion.

Accordingly the cap 42 is inserted through the incision 10 and threadedly engaged on the threaded end 41 of the stem 40 which forms a further component of the femoral implant. During the carrying out of the method shown in FIG. 9, it will be understood that the femoral head is still dislocated from the acetabulum.

Once the cap 42 has been secured to the stem 40, a hand-operated impactor device, for example a slaphammer 43, (FIG. 10), on the rod 37 is used to bring the cap 42 of the femoral implant down onto its desired position on the prepared femoral head, with the stem 37 moving downwards in the part of the canal 19 in the femoral head and neck.

Once the cap 42 has been brought to a position where it is fly engaged on the femoral head 14, as shown in FIG. 11, the impactor rod 37 is then unscrewed from the stem 40 and withdrawn through the incision 11. As shown in FIG. 11, the stem 40 thus remains screwed to the cap 42, being disposed in the upper part of the canal 19 within the femoral neck 13 and femoral head 14 in the same way as for a traditional stem fixing for a femoral implant component. Once the impactor rod 37 has been withdrawn, the part of the canal below the stem 40, which is open, is filled with morcellised bone graft 44. The femoral head, with its prosthesis is then reduced into the acetabulum which has its own new attached surface by way of the cup 32, and after closure of the surgical incisions 10 and 11, the resurfacing operation is complete. By use of the method described, i.e. a limited surgical approach (approaches), the amount of trauma to the patient is minimised, facilitating more rapid patient rehabilitation and reduced length of postoperative hospital stay.

Instead of the stem 40 being screwed to the cap 42, it could be integral therewith, its free end having the threaded socket 39 for engagement with part 38 of rod 37, or having alternative releasable connection means for connecting it with complementary means on the rod 37. This alternative femoral implant component could be inserted through an extended anterior, central or posterior incision at the hip joint, which extended incision is still smaller than the current single incision.

With the alternative form of femoral implant referred to, i.e. an unstemmed component comprising merely the cap 42, the impactor rod 37 would be a single component replacing the rod 37 and stem 40 shown in FIGS. 9 and 10, with the upper end of the impactor rod 37 extending out of the top of the femoral head and being screw-threaded to engage with a complementary internally threaded socket at the centre of the interior of the cap 42. Thus after the cap 42 is brought to is FIG. 11 position, the impactor rod would be unscrewed therefrom so that the whole of the canal extending through the femoral head, the femoral neck and the femur would thereafter be filled with bone graft 44 and there would be no stem connecting the cap 42 to the femoral head.

If an unstemmed cemented femoral resurfacing cap is considered, it is realised that use of a conventional cementing technique will not be possible due to limitation of space. With one known hip resurfacing method as set out in the introduction, the femoral component cap is filled with low viscosity cement and the component impacted into position thus pressurising cement into the peripheral cancellous bone of the prepared femoral head. However due to the limited access and the fact that the femoral prosthesis impactor rod has to be attached to the femoral resurfacing cap, low viscosity cement cannot just be poured into the femoral component, as it cannot be tilted with the prosthesis impactor rod attached, and the low viscosity cement will simply run out into the surgical wound and the inner aspect of the acetabular component, before the femoral component can be inserted. Accordingly for use of a cemented stemmed or unstemmed component, a totally different technique of cementing is therefore envisaged.

Taking firstly the simplest case of cementing an unstemmed femoral component cap, then the procedure will be as follows. Firstly the femoral prosthesis impactor rod for cemented use will be different, the rod needing to be cannulated so that ultra-low viscosity cement can be injected up the canal 19, in the inside of the femoral prosthesis impactor rod to reach the inside of the femoral prosthesis cap. Accordingly with this method, the cannulated impactor rod is attached to the femoral resurfacing cap by the previously mentioned screw connection. The femoral prosthesis is partly impacted until its parallel sides make contact with the parallel sides of the femoral head, thus creating a seal at the periphery of the femoral component. Two variations would be possible in design, one being that the canal within the impactor rod opens into the inside of the femoral component cap just before its connection to the cap, so that when ultra-low viscosity cement is injected, then the inside of the femoral component cap is filled with cement. The second possible design is one where within the substance of the femoral component cap a canal could be created which connects to the canal of the impactor rod and exits through the substance of the femoral component cap into the inner surface so that when low viscosity cement is injected, the inside of the femoral component cap is filled with ultra-low viscosity cement.

Once this cavity is filled with cement, the cement cannot run out because the seal has already been made at the periphery, and the hand-operated impactor rod is then used, as previously described, to bring the femoral prosthesis to its desired position, pressurising the cement into the peripheral femoral head cancellous bone.

A similar set-up can be imagined for the stemmed variety of cemented femoral component, whereby the stem of the femoral prosthesis has a canal also which either exits at the junction area between the stem and the cap or alternatively exits via a canal within the substance of the femoral component cap and thence into the cavity of the femoral head component.

FIGS. 12 and 13 show an alternative to the screw-threaded connections previously described and illustrated between a drive rod/impactor rod/stem and the interior of a cup-shaped component. Schematically shown in FIGS. 12 and 13 is a cup-shaped component 45, such as an acetabular reamer component, having a three legged spider-like member 46 releasably connected thereto to lie in the interior thereof. When used as in FIG. 7, the legs of member 46 engage the chamfer 29. The member 46 has a central circular section opening 47 which a tangentially arranged generally cylindrical projection 48 breaks into, as shown in FIG. 12.

The end of the cylindrical rod 49 which is to releasably connect with the member 46 has part cut away to form a flat surface 50, the amount cut away substantially corresponding to the amount the projection 48 breaks into the opening 47. As orientated in FIGS. 12 and 13 the flat is aligned with the projection, and the rod can thus be freely inserted into and removed from the opening. However the end of the rod is formed with a part-cylindrical groove 51 which breaks into the flat surface and is of a radius matching the projection 48. This allows the rod to be inserted into the opening as described and then rotated to receive part of the projection in the groove and lock the rod in the opening.

This illustrated form of releasable connection, which is an alternative to a screw thread, could also be used between the femoral implant component applicator stem and the stem of the stemmed femoral implant component, particularly when the stem of the femoral implant component is not integral with the cup-shaped part of said implant component.

1. A method of hip resurfacing comprising the steps of making a first incision at the hip joint, and making a second incision at an outer side of the thigh, inserting a stem part of an instrument, or an implant component applicator stem, through said second incision and along the inside of the femoral neck, and inserting an operative part of the instrument, and/or an implant component, through said first incision, the operative part being releasably connected to said stem part, and the implant component being releasably connected directly or indirectly to said stem, or acted upon by the or an instrument.

2. A method as claimed in claim 1, comprising preparing the femoral head to receive a femoral implant component and preparing the acetabulum to receive an acetabular implant component, said femoral and acetabular implant components being inserted through said first incision.

3. A method as claimed in claim 1, comprising inserting an acetabular reamer through said first incision, removably connecting said reamer to a stem of an instrument inserted through said second incision, the reamer being disposed over a prepared, resected femoral head, and reducing the femoral head and the acetabular reamer into the acetabulum for acetabular reaming to occur.

4. A method as claimed in claim 1, comprising initially making said first and second incisions, and then producing a canal which extends from said second incision through the femur and the femoral neck, and exits through the zenith of the femoral head.

5. A method as claimed in claim 4, comprising producing said canal by over-drilling a guide-wire initially inserted through said second incision into the lateral aspect of the femur and up into the femoral neck and head.

6. A method as claimed in claim 4, comprising inserting a drive rod into said canal through said second incision, inserting a sleeve cutter through the first incision, releasably securing the sleeve cutter to said drive rod, driving said drive rod, advancing said sleeve cutter onto the femoral head thereby resecting the periphery thereof, disconnecting the sleeve cutter from the drive rod, and removing them through said first and second incisions respectively.

7. A method as claimed in claim 4, comprising inserting a sleeve resection guide through said first incision, applying said guide to a peripherally resected femoral head, supplying a cutter, resecting an amount of zenith of the femoral head by means of said cutter, and removing said guide through said first incision.

8. A method as claimed in claim 4, comprising inserting a drive rod into said canal through said second incision, inserting a chamfer cutter through said first incision, releasably connecting the chamfer cutter to the drive rod, driving said drive rod, advancing said chamfer cutter onto a resected femoral head thereby to chamfer at least an uppermost part thereof, disconnecting the chamfer cutter from the drive rod, and removing said chamfer cutter through said first incision.

9. A method as claimed in claim 4, comprising inserting an acetabular reamer through said first incision, releasably connecting said actabular reamer to a drive rod received in said canal, driving said drive rod, advancing said reamer onto the acetabulum, and effecting reaming thereof.

10. A method as claimed in claim 9, wherein the acetabulum is successively reamed by releasably connecting to the drive rod each time one of a plurality of increasingly sized acetabular reamers, each of which is inserted into, and removed out of, said first incision.

11. A method as claimed in claim 10, wherein a prepared resected femoral head and a reamer disposed over it are together reduced into the acetabulum for acetabular reaming to occur.

12. A method as claimed in claim 4, comprising inserting through said first incision an acetabular implant component for fitting at a prepared resected acetabulum, inserting an inpactor rod into said canal through said second incision, inserting an impactor cap through said first incision to fit interiorally in the acetabular implant component, releasably connecting said rod and said cap, impacting said rod to fit the acetabular implant component at said prepared resected acetabulum, disconnecting said cap from said rod, and removing them through said first and second incisions respectively.

13. A method as claimed in claim 12, comprising holding said acetabular insert in said prepared acetabulum by means of a detachable holder.

14. A method as claimed in claim 12, comprising cementing said acetabular insert to said prepared acetabulum.

15. A method as claimed in claim 4, comprising inserting through said first incision a femoral implant component, releasably connecting said component to a rod received in said canal, impacting said rod to move the component onto a prepared, resected femoral head, disconnecting said component from said rod, and removing said rod through said second incision.

16. A method as claimed in claim 4, wherein the femoral implant component has a stem which remains in part of the canal, and attached to a head of the component, when the rod is removed.

17. A method as claimed in claim 15, during which the femoral head is dislocated from the acetabulum.

18. A method as claimed in claim 15, comprising filling all of the canal which is open with bone graft.

19. A method as claimed in claim 18, comprising after said filling, reducing the femoral head with said femoral implant component fixed thereto into the acetabulum with its implant component fitted thereto.

20. A method as claimed in claim 19, comprising the closing of said first and second incisions.

21. An instrument for use in hip resurfacing comprising a stem part and a releasably connectable operative head part, portions of the releasable connection being provided at or adjacent an end of the stem part and at said head part respectively.

22. An instrument as claimed in claim 21, wherein said operative head part is a sleeve cutter.

23. An instrument as claimed in claim 21, wherein said operative head part is a chamfer cutter.

24. An instrument as claimed in claim 21, wherein said operative head part is an acetabular reamer.

25. An instrument as claimed in claim 21, wherein said operative head part is an impactor cap.

26. An instrument as claimed in claim 21, wherein the releasable connection is a screw-thread.

27. An instrument as claimed in claim 21, wherein the stem part is releasably connectable to a member, separate from, but connected to said head part.

28. An instrument as claimed in claim 27, wherein said separate member is spider-like.

29. An instrument as claimed in claim 27, wherein said member has a central circular opening in which an end of said stem is intended to be received, the end of the stem being configurated so that it can be freely inserted into and out of the opening in one angular orientation, and locked in said opening by angularly moving it therein from said one angular orientation.

30. An instrument as claimed in claim 28, wherein the opening has a cylindrical projection therein and the end of the stem is provided with a flat and a groove extending therefrom, the stem being freely insertable into and removable from the opening when said flat is aligned with said projection, angular movement of the stem to bring the projection into the groove, locking the stem to said member.

31. A femoral implant component for use in hip resurfacing comprising a generally cup-shaped part having a part-spherical outer surface and connection means for directly or indirectly connecting the component, in use, with a rod/stem of an implant component applicator.

32. A component as claimed in claim 31, wherein the connection means is a screw-threaded socket.

33. A component as claimed in claim 32, wherein the socket is provided at the centre of the inside of the cup-shaped component.
